(19) United States
(12) Patent Application Publication
(10) Pub. No.: US 2021/0161402 A1
(43) Pub. Date: Jun. 3, 2021

(54) SYSTEM AND METHOD FOR EARLY PREDICTION OF A PREDISPOSITION OF DEVELOPING PREECLAMPSIA WITH SEVERE FEATURES

(71) Applicants: UNIVERSITY OF FLORIDA RESEARCH FOUNDATION, INCORPORATED, Gainesville, FL (US); CONVERGENT ENGINEERING, INC., Newberry, FL (US)

(72) Inventors: Neil Russell EULIANO, Gainesville, FL (US); Tammy Y. EULIANO, Gainesville, FL (US); Konstantinos MICHALOPOULOS, Gainesville, FL (US); Savyasachi SINGH, Gainesville, FL (US)

(21) Appl. No.: 16/632,671

(22) PCT Filed: Aug. 1, 2018
(86) PCT No.: PCT/US2018/044897

§ 371 Date: Jan. 21, 2020

Related U.S. Application Data
(60) Provisional application No. 62/539,781, filed on Aug. 1, 2017.

(51) Int. Cl. A61B 5/0205; A61B 5/00; A61B 5/1455; A61B 5/11; G16H 50/20; G06N 20/00

(52) U.S. Cl. CPC A61B 5/02055; A61B 5/7475; A61B 5/7267; A61B 5/14551; A61B 5/02125; A61B 5/1118; A61B 5/0022; G16H 50/20; G06N 20/00; A61B 5/4875

(57) ABSTRACT

A system and method for diagnosing and classifying preeclampsia-related conditions in a patient is provided. Also provided is a system and method for distinguishing preeclampsia-related conditions from other forms of hypertension that may be present in labor and delivery as well as distinguishing patients who will develop the more severe form of preeclampsia. The preeclampsia diagnosis and classification system utilizes non-invasive tests and comprises at least one sensor and a processor comprising a preeclampsia recognizer. In certain embodiments, the system further comprises a user interface.

### BACKGROUND

Preeclampsia is a major cause of maternal and neonatal morbidity and mortality around the world, responsible for approximately 76,000 maternal and 500,000 infant deaths per year (Preeclampsia Foundation, “About Preeclampsia,” (2012)). Its heterogeneous presentation complicates diagnosis and institution of therapy, while causing unnecessary treatment in many others. Left untreated, preeclampsia can rapidly and unexpectedly worsen to life-threatening hypertension, seizures, pulmonary edema and coagulation system effects. Early recognition of the symptoms, treatment of hypertension, prevention of seizures with magnesium and progression to delivery (the only cure, even if preterm) minimizes mortality. Recent studies of angiogenic factors as diagnostic tests hold promise, but at substantial cost. Currently there are no readily available, non-invasive tests to diagnose preeclampsia.

Preeclampsia affects 5-8% of pregnancies in the US, with its complications accounting for 18% of maternal deaths. Maternal and fetal morbidity present an additional, if immeasurable cost. The pathophysiology of preeclampsia remains an area of intense research, the outcome of which should lead to novel prevention and treatment strategies.

In the meantime there are methods to reduce morbidity and mortality such as blood pressure control, magnesium sulfate to prevent eclamptic seizures and delivery of the premature infant in a center with necessary capabilities. Diagnosis of preeclampsia in the previously normotensive patient presenting with typical symptoms (new-onset hypertension and proteinuria) is uncomplicated. However, nearly one-third of preeclamptics do not present so clearly (von D P et al. “Prediction of adverse maternal outcomes in preeclampsia: development and validation of the full PIERS model.” Lancet Jan. 15, 2011; 377(9761):219-27). In fact even in those with seizures (eclampsia), almost half (43%) were not previously diagnosed with both hypertension and proteinuria (Douglas K A, Redman C W. Eclampsia in the United Kingdom. BMJ Nov. 26, 1994; 309(6966):1395-400. PMCID:PMC2541348). Development of a low-cost, portable, reliable device to diagnose preeclampsia would reduce complications and mortality.

While many groups have investigated various ways to predict or detect preeclampsia, the vast majority of techniques require expensive equipment or laboratory tests. Of recent interest is angiogenic markers, primarily placental growth factor and soluble Fms-like tyrosine kinase-1 (Benton S J, et al. “Angiogenic factors as diagnostic tests for preeclampsia: a performance comparison between two commercial immunoassays.” Am. J Obstet. Gynecol. November 2011; 205(5):469-8). Unfortunately, cost and assay availability are primary limitations to ensuring diagnosis of preeclampsia via detection and/or quantification of such markers. Identification of the cardiovascular changes unique to preeclampsia may provide an alternative for diagnosis.

Additionally, there is a need for diagnosis of preeclampsia, distinguishing it from other forms of hypertension that may be present in labor and delivery as well as distinguishing parturients who will develop the more severe form of preeclampsia.

Maternal arterial characteristics in preeclampsia have been evaluated using non-invasive applanation tonometry in which a device, applied to the radial artery, extracts the pressure waveform; analysis of the reflecting waves infers vascular resistance. This device is expensive, requires training, and suffers from reproducibility issues, but the studies provide useful insight into the physiology. In a cross-sectional study of 69 normotensive and 54 preeclamptic pregnant women, Kaihura et al. detected a 20% difference in the carotid to femoral median pulse wave velocity; and a 10% difference between carotid and radial (Kaihura C et al. “Maternal arterial stiffness in pregnancies affected by preeclampsia.” Am. J Physiol Heart Circ. Physiol August 2009; 297(2):H759-H764). The group deduced an increase in maternal arterial stiffness with preeclampsia.

Similarly Arioz et al. studied 60 consecutive pregnant women in the third trimester of pregnancy with digital photoplethysmography and 24-hour ambulatory blood pressure (Arioz D T et al. “Arterial stiffness and dipper/nondipper blood pressure status in women with preeclampsia.” Adv. Ther. September 2008; 25(9):925-34). Thirty women were preeclamptic by standard criteria, a surprisingly high incidence. For this study, the group calculated the arterial stiffness index (SI) from the digital volume pulse (DVP) obtained with pulse oximetry. This study monitored changes in SI in preeclamptic patients. Unfortunately, this study failed to offer any suggestions for determining those patients likely to develop preeclampsia or those patients with non-symptomatic preeclampsia. Moreover, changes in SI alone do not necessarily provide an accurate means for determining those patients likely to develop preeciampsia or diagnosing those patients with non-symptomatic preeclampsia.

Described in 2000 by Millasseau et al., the first derivative with respect to time of the DVP is used to identify the inflection point (similar to the dichrotic notch in an arterial waveform) (Millasseau S C et al. “Contour analysis of the photoplethysmographic pulse measured at the finger.” J. Hypertens. August 2006; 24(8):1449-56). The time between the systolic peak and this notch is calculated and used to derive the SI as body height/ΔT. Arioz et al. (Ibid.) identified a 50% increase in SI (5.9±0.8 m/s vs. 8.8±1.2) with preeclampsia. Most recently, Avni et al. examined 100 pregnant patients including preeclamptic, chronic hypertensive, and normotensive parturients. Their findings agree with those above, identifying an increase in aortic stiffness, as assessed by pulse wave analysis with applanation tonometry (Avni B et al. “Aortic stiffness in normal and hypertensive pregnancy.” Blood Press February 2010; 19(1):11-5). These studies used devices impractical for routine use in clinics, especially by less trained personnel. Noninvasive applanation tonometry is performed in a device applied to the radial artery that extracts the pressure waveform. This device is expensive, requires training, and suffers from reproducibility issues, but the studies provide useful insight into the physiology.

In another example, a method for monitoring preeclampsia involves analysis of cardiovascular oscillations noninvasively via a finger cuff (H Malberg et al., “Analysis of cardiovascular oscillations: A new approach to the early prediction of pre-eclampsia,” Chaos 17, 015113 (2007)). According to the Malberg et al. system, the finger cuff continuously monitors blood pressure and extracts time series of beat-to-beat intervals, and systolic and diastolic blood pressures (Portapres device, BMI-TNO). The Malberg et al. system is rather complex and illustrated in FIG. 9. Malberg et al. observed 96 patients with abnormal uterine perfusion identified by doppler sonography, 24 of whom eventually developed preeclampsia. They utilized a variety of entropy measures and statistical methods to analyze heart rate (HR) and blood pressure variability, etc.

Another method (Khalil A. et al. (2009) “Pulse Wave Analysis in Normal Pregnancy: A Prospective Longitudinal Study.” PLoS ONE 4(7): e6134. doi:10.1371/journal.pone.0006134) involves pulse wave analysis. Pulse wave analysis provides valuable information in hypertension and vascular disease. Khalil et al. used a tonometer to measure arterial pulse waves and, following pulse wave analysis, evaluated changes in pulse wave analysis parameters to investigate whether these parameters are affected by ethnicity. Unfortunately, tonometers are expensive and difficult to use, with reliability and repeatability issues.

Khalil A. et al. (“Pulse wave analysis: a preliminary study of a novel technique for the prediction of pre-eclampsia.” BJOG 2009; 116:268-277) also investigated whether first-trimester arterial pulse wave analysis can predict preeclampsia. In this study, 11-14 weeks of gestation pulse waves were measured with tonography. Arterial PWA was performed as follows: the radial artery was gently compressed with the tip of the tonometer at the site of maximal pulsation. This tonometer contains a micromanometer that provides a very accurate recording of the pressure within the radial artery (Millar Instruments, Houston, Tex., USA). Unfortunately, as indicated above, tonometers are expensive and difficult to use, with reliability and repeatability issues.

In a further study, radial artery applanation tonometry was utilized (Spasojevic et al. “Peripheral arterial pulse wave analysis in women with pre-eclampsia and gestational hypertension,” BJOG: an International Journal of Obstetrics and Gynaecology, November 2005, Vol. 112, pp. 1475-1478). Women in the third trimester of pregnancy with newly developed preeclampsia (PE) (n=27) or gestational hypertension (GH or HTN) (n=33) were studied by radial artery applanation tonometry. Spasojevic et al. determined hypertension was of equal severity in PE and GH and concluded measurement of Augmentation Index (AI) gives clear separation of established PE both from normal pregnancy and from uncomplicated GH. As indicated above, tonometers are, unfortunately, expensive and difficult to use, with reliability and repeatability issues.

Applicant has identified a need to develop reliable, inexpensive and clinically useful parameters for predicting preeclampsia. Being able to identify pregnant women at risk for developing preeclampsia with severe features could permit the use of therapy and enhanced patient care so as to prevent severe preeclampsia development.

### BRIEF SUMMARY

The subject invention provides an inexpensive, non-invasive system and method for predicting and/or determining a predisposition of developing preeclampsia in a patient. While the disease can begin benignly enough with a headache, severe features may arise such as life-threatening hypertension, seizures, pulmonary edema, kidney or liver damage, lung damage, and coagulation system effects can occur rapidly and unexpectedly. Even in developed countries, complications and deaths occur as a result of preeclampsia. Therefore, early recognition of the symptoms, treatment of hypertension, prevention of seizures and progression to delivery (the only cure, even if preterm) minimizes mortality. Unfortunately many low-income countries lack access to the proper test (blood pressure and urine protein testing) to even diagnose preeclampsia once it manifests, let alone predict whether severe features are likely to occur in the future. In addition to operating as an early-warning prediction system, the subject invention detects preeclampsia after onset (and, in certain instances, prior to detection of conventional symptoms associated with preeclampsia), predicts severe maternal outcomes for women with early signs of preeclampsia, and facilitates treatment and/or delivery or transfer planning.

A sensor device is disclosed that includes sensors adapted to be worn on a patient's body. The sensors include those that generate information indicative of detected physiological parameters of the patient. In one embodiment, a sensor device is provided comprising a pulse oximeter probe and at least one ECG sensor, wherein the sensors generate data indicative of photoplethysmographic (PPG) measurements and electrocardiogram (ECG) signal(s), respectively. The sensor device can be produced from inexpensive and/or reusable sensor technologies. In certain embodiments, the sensor device is portable and/or wearable.

The sensor device can further include a housing adapted to be worn on a patient's body, wherein the housing supports the sensors or wherein at least one of the sensors is separately located from the housing. The sensor device may further include a flexible body supporting the housing having first and second members that are adapted to wrap around a portion of the patient's body. The flexible body may support one or more of the sensors. The sensor device may further include wrapping means coupled to the housing for maintaining contact between the housing and the patient's body, and the wrapping means may support one or more sensors.

The sensor device can include any one or more of the following: a processor that receives at least a portion of data generated by the sensors and is adapted to generate derived data related to the detection and/or prediction of preeclampsia; a display for communicating information regarding the data collected by the sensor device; a user interface. In one embodiment, illustrated in FIG. 2, the sensor device is a portable or wearable device provided on a wrist strap.

The invention is also directed to a system for predicting and/or diagnosing preeclampsia in a patient. The system of the invention comprises a sensor device, a processor adapted to generate derived data from the information provided by the sensor device, and a user interface for reporting the likelihood of current or future preeclampsia. The sensor device can include the processor or the processor may alternatively be external to the sensor device. The reports from the user interface can be provided to the patient and/or to clinical personnel. The system can be customized based on local clinical infrastructure and cultural differences and can be programmed to provide follow-up and/or therapy advice, including reprogramming as recommendations change. Furthermore, data collection to better understand the effectiveness of various treatments is also feasible. The system could also transmit data to a central server which performs the required processing to interpret the data using the latest algorithms. The results of the processing along with location- or cultural-specific therapy recommendations could then be transmitted back to the device, the user's cell phone, or other communication device.

Advantages of the invention include one or more of the following. The system allows patients and/or clinicians to conduct a low-cost, comprehensive, real-time monitoring for preeclampsia. Use of the subject invention can result in diagnosis and treatment of preeclampsia and, in some cases, predict preeclampsia before symptoms are detected. Because the system is non-invasive and, in certain embodiments, has no disposable parts, its cost per patient is very small, perhaps a penny per patient test or less.

The subject invention is simple to use and modular. For example, the sensor device can be built in many easy to use form factors including an armband that simply straps around the wrist of a patient. After a few minutes of data collection, a display will indicate the likelihood of present or future onset of preeclampsia. Additionally, the information can be sent via multiple methods to a computer, website, external database, or other location for analysis, storage, and/or further processing. Untrained or minimally trained clinical personnel (or the patient) can use the system.

The system provides real time and point of care prediction and/or detection of preeclampsia. There is no required lab work or any delay in test result reporting. The system is placed on the patient and within a few minutes provides the results of the test.

In particular, the system is easy to maintain There is no calibration, chemical testing, or other complicated methods necessary. Only recharging of the battery or application of power is required for the sensor device.

The system of the invention preferably comprises a portable and/or wearable sensor device. The sensor device may be small and easily worn by the patient and can non-invasively capture data on plethysmographic waveform and ECG to report detection and/or prediction of preeclampsia. Preferably, the sensor device is a cuff that can be worn on the arm or the wrist.

In one embodiment, the system comprises a sensor device that captures data on plethysmographic waveform and single-channel ECG to non-invasively detect preeclampsia, as well as to differentiate between mild and severe preeclampsia. The subject system may be used in labor & delivery suites and emergency departments for early diagnosis of preeclampsia and initiation of magnesium therapy where indicated.

The subject system facilitates the diagnosis of preeclampsia, distinguishing it from other forms of hypertension that may present in labor and delivery. This enables magnesium therapy to be initiated appropriately, in only those patients who will benefit. The system also identifies parturients at prenatal visits who are at high risk of developing preeclampsia, and distinguishes those who will develop the more severe form. Such a device enhances patient care by:
allowing transfer of such patients to an appropriate-level provider (e.g. home delivery becomes less desirable).
encouraging directed education of the identified high-risk patient regarding warning signs and increased frequency of blood pressure monitoring.
enabling healthcare providers to plan for more frequent evaluations of the fetus and the potential for a preterm delivery. For example, if severe complications are predicted, (a) more frequent prenatal visits and observation of fetal growth may be indicated, (b) antenatal steroids for lung maturation may be considered, and (c) development of contingency plans for delivery at a center with a neonatal intensive care unit (NICU) and availability of blood products should HELLP syndrome (a clotting disorder) develop.
facilitating research protocols into prevention and treatment strategies that are best implemented in a population of known risk, e g administration of dietary supplements. This could be investigated at reasonable cost in the subgroup of patients identified with this technology.

With early prediction capabilities, the subject invention can be part of routine screening in medical clinics that offer prenatal care. Once preeclampsia is identified, the system could improve outcomes for both patient/mother and fetus by enabling (1) directed patient education, (2) increased prenatal monitoring, (3) administration of supplements that may reduce preeclampsia severity, and (4) delivery planning, including transportation to an appropriate facility. Furthermore, the system may include real-time updates on recommendations from American Congress of Obstetricians and Gynecologists (ACOG), and could suggest possible study protocols. The system also has a large potential for use in research of preeclampsia and treatments. For example, use of the system as an accurate screening device in clinical trials assessing treatments for preeclampsia could provide significant cost and resource savings.

### BRIEF DESCRIPTION OF THE DRAWINGS

Having thus described certain example embodiments of the present disclosure in general terms, reference will now be made to the accompanying drawings, which are not necessarily drawn to scale, and wherein:

FIG. 1 is an exemplary overview of a system that can be used to practice embodiments of the present invention;

FIG. 2 illustrates an embodiment of the invention consisting of a wrist worn sensor device in accordance with some embodiments herein.

FIG. 3 illustrates an example client device in accordance with some embodiments discussed herein;

FIG. 4 illustrates an example preeclampsia diagnosis and classification system in accordance with some embodiments discussed herein;

FIG. 5 illustrates a flow diagram of an example preeclampsia diagnosis and classification system in accordance with some embodiments discussed herein;

FIG. 6 illustrates a plethysmogram waveform, examples of the extracted features, and its second derivative, the acceleration plethysmogram. Crest time, delta T, pulse width and AI: augmentation index;

FIG. 7 illustrates a characteristic curve for (A) normotensive controls (negative class) versus severe preeclampsia (positive class);

FIG. 8 is an example output produced by various embodiments of the present invention;

FIG. 9 illustrates one embodiment of the prior art; and

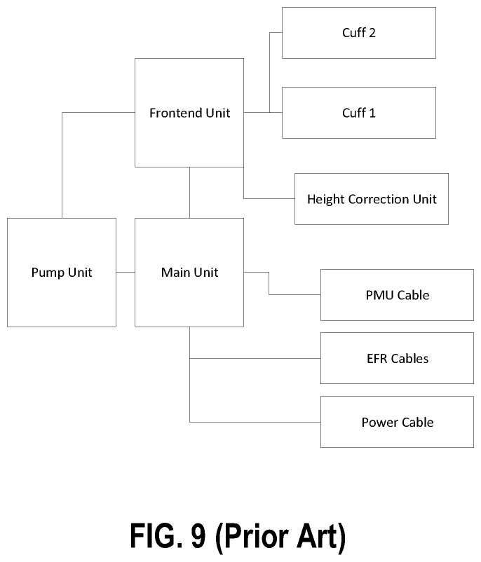

FIG. 10 illustrates a typical ECG and PPG waveform with features of each waveform and timing parameters.

### DETAILED DESCRIPTION

A system and method for detecting preeclampsia in a patient is provided. Also provided is a system and method for diagnosing preeclampsia in a patient prior to the detection of conventional symptoms or clinical signs associated with preeclampsia. Conventional symptoms associated with preeclampsia include, but are not limited to, swelling, abdominal pain, seizures, sudden weight gain, headaches and changes in vision. Typical clinical signs include hypertension, protein in the urine, and hyperreflexia. The preeclampsia detection system of the invention comprises a sensor device and a processor comprising a preeclampsia recognizer. In certain embodiments, the system further comprises a user interface.

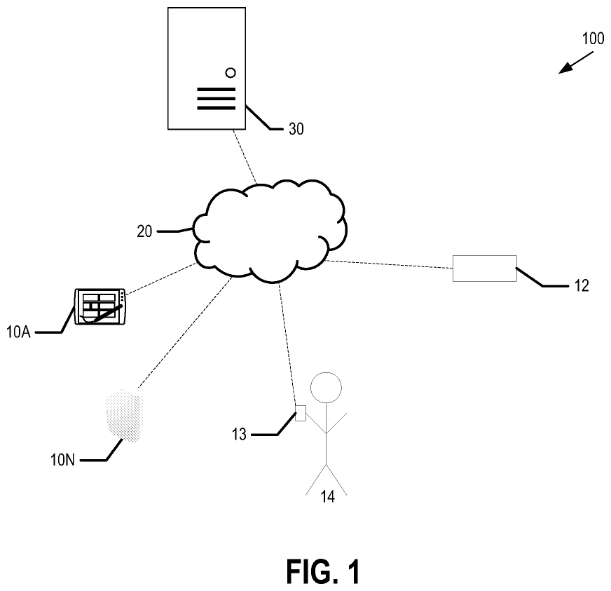

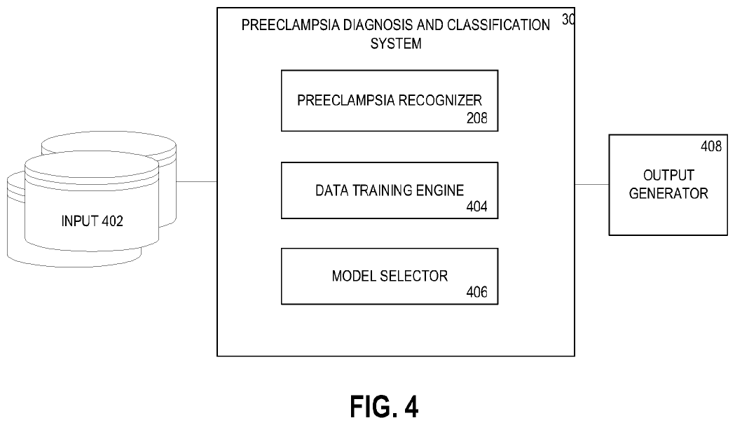

FIG. 1 provides an illustration of an exemplary embodiment of the present invention. FIG. 1 shows system 100 including an example network architecture for a system, which may include one or more devices and sub-systems that are configured to implement some embodiments discussed herein. For example, the system 100 may include preeclampsia diagnosis and classification system 30, which can include, for example, a server or database, among other things (not shown). The preeclampsia diagnosis and classification system 30 may include any suitable network server and/or other type of processing device. In some embodiments, the preeclampsia diagnosis and classification system 30 may determine and transmit commands and instructions for training models, generating optimal prompting strategies to maximize a user's medical adherence (e.g., a user's predicted diagnosis of preeclampsia with severe features) to client devices 10A-10N using data stored via a database (not shown) which may be stored as a part of and/or in communication with one or more client devices 10A-10N and/or the preeclampsia diagnosis and classification system 30. The database includes information accessed and stored by the client device 10 to facilitate the operations of the preeclampsia diagnosis and classification system 30 (shown in FIG. 4).

Preeclampsia diagnosis and classification system 30 can communicate with one or more client devices 10A-10N and/or other computing entities via communications network 20, and a plurality of client devices 10A-10N may communicate with one another and/or other computing entities via the network 20. In this regard, communications network 20 may include any wired or wireless communication network including, for example, a wired or wireless local area network (LAN), personal area network (PAN), body area network (BAN), metropolitan area network (MAN), wide area network (WAN), or a serial communication connection, standard serial buses such as, for example, RS232 or universal serial bus (USB), Serial Peripheral Interconnect bus (SPI), Inter-integrated Circuit bus known as the I²C (read “I-squared C”) bus, or the like, as well as any hardware, software and/or firmware required to implement it (such as, e.g., network routers, etc.). For example, communications network 20 may include a cellular telephone, an 802.11, 802.16, 802.20, and/or WiMax network. Further, the communications network 20 may include a public network, such as the Internet, a private network, such as an intranet, or combinations thereof, and may utilize a variety of networking protocols now available or later developed including, but not limited to TCP/IP based networking protocols. For instance, the networking protocol may be customized to suit the needs of the group-based communication interface. In some embodiments, the protocol is a custom protocol of JSON objects sent via a Websocket channel. In some embodiments, the protocol is JSON over RPC, JSON over REST/HTTP, and the like.

Client devices 10A-10N and/or preeclampsia diagnosis and classification system 30 may each be implemented as one or more computers, computing entities, desktop computers, mobile phones, tablets, phablets, notebooks, laptops, distributed systems, gaming consoles (e.g., Xbox, Play Station, Wii), watches, glasses, iBeacons, proximity beacons, key fobs, radio frequency identification (RFID) tags, ear pieces, scanners, televisions, dongles, cameras, wristbands, wearable items/devices, items/devices, vehicles, kiosks, input terminals, servers or server networks, blades, gateways, switches, processing devices, processing entities, set-top boxes, relays, routers, network access points, base stations, the like, and/or any combination of devices or entities adapted to perform the functions, operations, and/or processes described herein. The depiction in FIG. 1 of “N” client devices is merely for illustration purposes. Any number of users and/or client devices 10 may be included in the system for accessing and/or implementing aspects of the preeclampsia diagnosis and classification system 30 discussed herein (e.g., via one or more interfaces). In one embodiment, the client devices 10A-10N may be configured to display or provide a medication prompting interface on a display of the client device for viewing, creating, editing, and/or otherwise interacting with one or more automated-prompting notifications, which may be provided or pushed by the preeclampsia diagnosis and classification system 30 (and may be stored locally at one or more client devices 10A-10N). According to some embodiments, the preeclampsia diagnosis and classification system 30 may be configured to cause display or presentation of an interface for viewing, creating, editing, and/or otherwise interacting with one or more automated-prompting notifications. In yet another embodiment, the preeclampsia diagnosis and classification system 30 may be configured to cause display or presentation of an interface for viewing a predicted likelihood of preeclampsia with severe features.

As indicated above, the client devices 10A-10N may be any computing device as defined above. Electronic data received by the preeclampsia diagnosis and classification system 30 from the client devices 10A-10N may be provided in various forms and via various methods. For example, the client devices 10A-10N may include desktop computers, laptop computers, smartphones, netbooks, tablet computers, wearables, and the like. In embodiments where a client device 10A-10N is a mobile device, such as a smart phone or tablet, the client device 10A-10N may execute an “app” such as the preeclampsia diagnosis and classification application to interact with the preeclampsia diagnosis and classification system 30. Such apps are typically designed to execute on mobile devices, such as tablets or smartphones. For example, an app may be provided that executes on mobile device operating systems such as iOS®, Android®, or Windows®. These platforms typically provide frameworks that allow apps to communicate with one another and with particular hardware and software components of mobile devices. For example, the mobile operating systems named above each provide frameworks for interacting with location services circuitry, wired and wireless network interfaces, user contacts, and other applications. Communication with hardware and software modules executing outside of the app is typically provided via application programming interfaces (APIs) provided by the mobile device operating system.

Additionally or alternatively, the client device 10A-10N may interact with the preeclampsia diagnosis and classification system 30 via a web browser. As yet another example, the client device 10A-10N may include various hardware or firmware designed to interface with the preeclampsia diagnosis and classification system 30.

A plurality of sensors (which in certain embodiments may be a part of one or more client devices 10A-10N) provide information to the preeclampsia diagnosis and classification system 30 (e.g., via network 20). For example, the sensors may provide tracking and monitoring maternal and fetal well-being, medication adherence sensors, blood pressure, weight sensors, motion sensors, power sensors for various devices (e.g., a treadmill, an exercise bicycle, and/or the like), and/or the like. It should be understood that these sensors, as well as other sensors discussed herein, are provided merely as examples, and any sensors that may be indicative of the health, location and/or activity of a user may be provided.

In some embodiments, the preeclampsia diagnosis and classification system 30 may be configured to be in communication with the one or more client devices 10A-10N to establish a cloud-based, device-based, and/or or fog-based (e.g., a networked system within a home, building, business, and/or the like, edge device, fog device or full public cloud) system for a user. Moreover, the preeclampsia diagnosis and classification system 30 may further offer a hybrid architecture to enable opt-in community-based feedback, transfer learning of disease-specific medical diagnosis strategies, and dietary and activity-based recommendations. The plurality of sensors may be embodied by Internet of Things (IoT) devices. In some example embodiments, one or more ambient/personal computing devices 13 (portable and/or wearable computing devices which may themselves constitute one type of client device 10) may also provide context information that may be utilized by the prompting system. The one or more ambient/personal computing devices may comprise motion sensors, home activity sensors, phone accelerometers, phone Wi-Fi readings etc. As depicted in FIG. 1, user 14 may interact with any of the client devices 10A-10N, and/or other devices 12, which in turn provide information to the preeclampsia diagnosis and classification system 30, or other computing entities storing data for predicting and diagnosing preeclampsia with severe features, generating and/or implementing an optimal prompting model/strategy to maximize a user's medical adherence. The other devices 12 may themselves be client devices 10A-10N that include additional components, such as an optional medication tracker/dispenser which may provide information as to whether the user 14 took his or her preeclampsia medication.

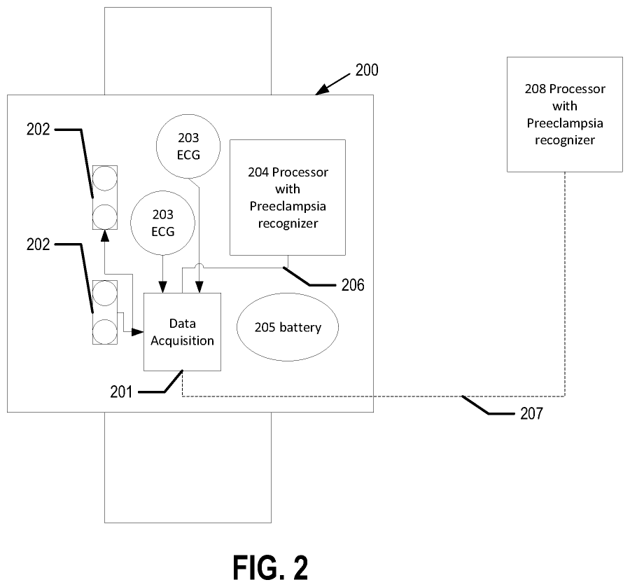

FIG. 2 shows an exemplary sensor device 200 (which in certain embodiments may be a part of one or more client devices 10A-10N and/or 13). The sensor device can operate in a home, clinic or hospital. In certain embodiments, the sensor device comprises one or more sensors situated together as a single unit to be non-invasively worn by or applied to a patient. In a related embodiment, the one or more sensors are situated within one or more housing units or devices. One embodiment of the sensor device comprises a simple wrist/arm band that is held in place via a watch-band, elastic band or Velcro strap, wherein situated on the device or band are one or more sensors. Because the intelligent algorithms of the system of the invention require only a single photoplethysmography (PPG) channel and a single electrocardiogram (ECG) channel, the sensors can comprise optical transducer(s) 202 and electrode sensor(s) 203. Preferably, two or more electrodes and one or more optical transducers are used.

The sensor device 200 may include various hardware or firmware comprising (or that interface with) the preeclampsia diagnosis and classification system 30. To this end, certain embodiments of the sensor device 200 comprise certain components of the preeclampsia diagnosis and classification system 30. As shown in FIG. 2, the example sensor device 200 includes a processor with a preeclampsia recognizer 204 (whereas the other components of the preeclampsia diagnosis and classification system 30 remain hosted in a separate device). However, as shown by the dotted lines in FIG. 2, an alternative embodiment may host the processor with preeclampsia recognizer separately, in which case the sensor device 200 is configured to communicate with those separately disposed components. When hosted by the sensor device 200, the preeclampsia recognizer is programmed to locally analyze patient data along with PPG and ECG signals using one or more models that are trained by the preeclampsia diagnosis and classification system 30. Alternatively, where the processor and preeclampsia recognizer 208 are hosted separately by the preeclampsia diagnosis and classification system 30, any patient data gathered by the sensor device 200 may be provided via a transmission channel 207 for analysis, and the output of the preeclampsia recognizer may be returned to the sensor device 200 by the transmission channel 207 (and similar exchanges of data may be facilitated by corresponding transmission channels between one or more other client devices 10A-10N and the preeclampsia diagnosis and classification system). Whether hosted by the sensor device 200 or located remotely in the preeclampsia diagnosis and classification system 30, the preeclampsia recognizer 208 can comprise one or more classification or models (for the detection, diagnosis, and prediction of different classes of preeclampsia). Additionally the sensor device 200 may be configured or programmed to periodically or continuously transmit data for future use. For instance, data collected by the sensor device 200 may be received by the preeclampsia diagnosis and classification system 30 and, once an authoritative diagnosis is made regarding the patient, the data may be incorporated into a corpus of training data and used by the preeclampsia diagnosis and classification system 30 for iteratively training one or more of the models utilized by the preeclampsia recognizer. In embodiments where an instance of the preeclampsia recognizer is disposed on the sensor device 200, then upon such iterative training of the preeclampsia recognizer by the preeclampsia diagnosis and classification system 30, updated model information may be transmitted by the preeclampsia diagnosis and classification system 30 to the sensor device 200 for updating the local instance of the preeclampsia recognizer.

In one embodiment, the sensor device 200 includes a minimal user interface indicating that the device is operating and how much battery life is remaining. In one embodiment, the preeclampsia recognizer 208 resides in a mobile phone that communicates wirelessly 207 via Bluetooth to the sensor device 200 and displays the preeclampsia recognizer outputs, therapy recommendations, fitness recommendations, and other outputs as described later.

An optical transducer can be a sensor comprising a light source and a photo-detector. The light source can be light-emitting diodes (LED) that generate red (λ=about 630 nm) and/or infrared (λ=about 900 nm) radiation, for example. The light source and the photo-detector are slidably adjustable and can be moved along the wrist/arm band to optimize beam transmission and pick up. As the heart pumps blood through the patient's finger, blood cells absorb and transmit varying amounts of the red and infrared radiation depending on how much oxygen binds to the cells' hemoglobin. The photo-detector detects transmission at the predetermined wavelengths, for example, red and infrared wavelengths, and provides the detected transmission to a pulse-oximetry circuit, which may also be located on the wrist/arm band. The output of the pulse-oximetry circuit is digitized into a time-dependent optical waveform (plethysmographic waveform), which is then sent back to the pulse-oximetry circuit for further analysis (e.g., by the processor) and/or further transmission (e.g., to the display). Although standard pulse-oximetry uses two frequencies of light to determine the amount of oxygenated hemoglobin, only one frequency of light is required to create a waveform of blood flow (plethysmography).

The sensor device can include at least one electrode sensor that enables differential ECG to be measured. Contemplated electrode sensors include, but are not limited to, disposable sensors (including sensors that are without gel or pregelled), reusable disc electrodes (including gold, silver, stainless steel, or tin electrodes), headbands, saline-based electrodes, impedance, radio frequency (RF), and acoustic sensors. Contemplated sensors include those used for monitoring electrocardiography (ECG/EKG); electroencephalography (EEG); electromyography (EMG); electronystagmography (ENG); electro-oculography (EOG), printed circuit sensors, electroretinography (ERG), bioimpedance sensors (RF or otherwise) and stethoscope sensors.

The electrical signal derived from an electrode is typically 1 mV peak-peak. In certain embodiments, an ECG amplifier (e.g., a one-channel ECG amplifier or differential amplifier) is provided to amplify the electrical signal by about 100 to about 1,000 times as necessary to render this signal usable for detection.

The sensors of the sensor device can be removable. Further, the sensors can be passive (such as a reader) and store information 201. Alternatively, or in addition, the sensors can transmit information (e.g., to a processor for analysis purposes).

The sensor electronics and power source of a sensor device are preferably small. The power source can be any portable power source capable of fitting on the sensor device. According to some embodiments, the power source is a portable rechargeable lithium-polymer or zinc-air battery 205. Additionally, portable energy-harvesting power sources can be integrated into the sensor device and can serve as a primary or secondary power source. For example, a solar cell module can be integrated into the sensor device for collecting and storing solar energy. Additionally, piezoelectric devices or microelectromechanical systems (MEMS) can be used to collect and store energy from body movements, electromagnetic energy, and other forms of energy in the environment or from the patient. A thermoelectric or thermovoltaic device can be used to supply some degree of power from thermal energy or temperature gradients. In some embodiments, a cranking or winding mechanism can be used to store mechanical energy for electrical conversion or to convert mechanical energy into electrical energy that can be used immediately or stored for later.

In one embodiment, the sensor device comprises at least one optical transducer, a pulse-oximetry circuit, at least one electrode, and a one-channel ECG amplifier that is provided in an electronic sensor assembly. The electronic sensor assembly is preferably small in size (approximately 2″×3″) and can be powered by two watch batteries or similar rechargeable technology. As such, this system is very small and can be wearable or portable.

In a related embodiment, the sensor device is a simple armband that contains two metal electrodes (similar to exercise watches or equipment) and one or more optical transducers. More than one optical transducer (photodetector and LED) may be provided on the armband, particularly those optical transducers that are very small and inexpensive, to ensure robust data collection across different band locations and arm sizes.

Alternatively, the system of the invention may comprise more than one sensor device. For example, the preeclampsia detection system can include a sensor device comprising one or more electrodes and another sensor device comprising one or more

PPG sensors. In one embodiment, the system comprises a standard finger pulse oximeter and simple ECG sensor placed anywhere on the body. In a related embodiment, multiple ECG sensors are provided on the maternal abdomen. Information from the electrodes on the maternal abdomen can be used not only to detect and/or predict preeclampsia but also for antepartum and/or intrapartum maternal fetal monitoring as described in U.S. Pat. No. 7,333,850, which is incorporated herein by reference in its entirety. Alternatively, the preeclampsia detection system may include the electrode ECG sensors and interface cable as described in U.S. Pat. No. 7,828,753, which is incorporated herein by reference in its entirety.

A signal conditioning front-end of the preeclampsia detection system amplifies the low level ECG bioelectric signals coming from the electrodes and provides low-impedance signals to a data acquisition module, which can be connected to or be a part of a processor. Active common mode noise suppression is used to reduce or eliminate 60 Hz electric power line noise typically present in signals from human body surface electrodes. The data acquisition module is designed with a low-power and low-noise 24-bit analog-to-digital converter (ADC). This 24-bit ADC provides a very large dynamic range that eliminates input saturation with high level muscle contraction signals, and has very high signal resolution, passing an accurate low-noise signal to the system processor (initially on the smartphone/PC, eventually an embedded processor in the armband). The system processor is used to process the ECG and PPG data streams acquired by the ADCs.

The sensor device preferably implements continuous ECG recording and collection of pulse oximetry sensor output waveforms (photoplethysmography, PPG) from various locations on a patient's body. Those locations include, but are not limited to, the finger, wrist, ear, nose, cheek, forehead, chest, abdomen etc. of the patient. For example, an array of sensors may be provided for the abdomen, where the array has a low spatial resolution.

In certain embodiments, the system comprises a user interface. The user interface can be a personal or tablet computer, a cell phone monitor, a PDA monitor, a television, a projection monitor, a visual monitor on the sensor device, or any method of visual display. One example user interface that may be used in the system is a low power liquid crystal display (LCD) or similar display on the armband.

Signal data from the sensor device(s) (e.g., PPG and ECG signals) are transmitted 206 to a processor. The data can be transmitted periodically or at a later time. This delayed transmission may, without restriction, be utilized to improve battery life by transmitting data transiently, instead of continuously; or to allow for patient monitoring during disconnection from the sensor device.

The processor 204 of the preeclampsia detection system is a device that performs any one or more of the following functions: (1) it stores the signals to memory, such as a flash or SRAM, for subsequent analysis; (2) it stores a number of signals to memory and subsequently transmits them, wired or wirelessly, to a remote computer for preeclampsia detection as described herein and/or display, such as display in real time; or (3) it processes the signals using a software module as described herein to detect preeclampsia in a patient. A variety of microprocessors or other processors may be used herein.

In one embodiment, a wireless signal transmitter 207 may be utilized between the sensor device(s) and the processor. The wireless signal transmitter can include a data storage device (such as a magnetic hard drive, flash memory card, and the like). Preferably, the wireless signal transmitter includes communications protocols for data representation, signaling, authentication, and error detection that required to send information over a wireless communications channel (i.e., a specific radio frequency or band of frequencies such as Wi-Fi, which consists of unlicensed channels 1-13 from 2412 MHz to 2484 MHz in 5 MHz steps). The wireless signal transmitter is preferably located on or near the sensor device(s). For example, the wireless signal transmitter can be attached to a housing on an armband of the sensor device. Many wireless transmission communications protocols exist and are applicable to the wireless signal transmitter/receiver of this invention, including Bluetooth, Wi-Fi, Zigbee, wireless USB, etc. The wireless transmission of information from the wireless signal transmitter to the wireless signal receiver could be in digital format or in analog format.

In certain embodiments, the wireless signal transmitter (and/or wireless signal receiver) includes an internal power source (i.e., batteries, and the like). Alternatively, the wireless signal transmitter (and/or wireless signal receiver) does not require an internal power source. This can be accomplished with a variety of energy harvesting or wireless power transmission methods such as harvesting of heat, movement, electrical signals from the environment, or inductive coupling. In one embodiment, this is accomplished by using an antenna to convert radiated or inducted power into usable energy for the transmission of the desired signals. For example, the wireless signal transmitter can be an antenna that is commonly used in radio frequency identification tags (or RFID tags), where minute electrical current induced in the antenna by an incoming radio frequency signal provides just enough power for an integrated circuit (IC) in the RFID tag to power up and transmit a response (for example, to a wireless signal receiver of the invention).

In one embodiment, the processor executes one or more software modules to analyze signals from the sensor device. More preferably, the processor is configured to run the preeclampsia recognizer 208 that is used to analyze PPG and ECG signals. For example, PPG and ECG signals can be used as input to a preeclampsia recognizer. A preeclampsia recognizer can comprise one or more classification, prediction, or other models (for the detection and/or prediction of preeclampsia). Such classifiers include, but are not limited to, simple clustering analysis and logistic regression models. Nonlinear models are also envisioned due to their classification and prediction performance, including but not limited to:
Support Vector Machines Similar to Radial Basis Function Network, this type of model separates the classes with high-dimensional hyper plane using the samples nearest the decision surface to maximize the margin.
Neural Network. Although traditionally a black box modeling tool, neural networks afford an increase in the degrees of freedom to model the aforementioned data non-linearly.
Information theoretic methods. Using these may help in modeling features that are non-Gaussian.
State-spaced methods. These models can identify hidden state information present in the data. Exploiting the temporal-state information may increase performance beyond our static classifier. The Kalman filter (continuous state-space) and Hidden Markov Model (HMM) are two such models that will be implemented.

In one embodiment, the preeclampsia recognizer is a statistical analyzer such as a neural network that has been trained to detect preeclampsia, detect severe aspects of preeclampsia, detect mild aspects of preeclampsia, and/or detect hypertension. The neural network can be a back-propagation neural network, for example. In this embodiment, the statistical analyzer is trained with training data where certain signals are determined to be undesirable for the patient. For example, the patient's desirable pattern of PPG and ECG signals or features should be within a well-established range, and any values outside of this range are flagged by the preeclampsia recognizer as a preeclampsia condition. Once the preeclampsia recognizer is trained, the data received by the processor can be appropriately scaled and processed.

In certain embodiments, the preeclampsia recognizer is trained from patient data to optimally separate a variety of patient scenarios, including: preeclamptics from non preeclamptics, mild versus severe preeclamptics, differentiation of preeclamptics from other forms of hypertension such as gestational hypertension, patients likely to eventually have preeclampsia symptoms. In a related embodiment, the preeclampsia recognizer is a Radial Basis Function Network (RBF) with a linear output to discriminate/detect preeclamptics versus controls.

In certain related embodiments, the patient data feature set consists of parameters from four different physiologic classes: A) heart rate, B) pulse transit time (PTT, correlates with blood pressure), C) augmentation indices, and D) oximetry. Multiple parameters from each class capture different representations of the fundamental data (e.g., heart rate or PTT variability), and combinations of parameters are also derived (e.g., change in PTT per change in heart rate). Using the different covariates, a high-dimensional feature vector is assembled as input into the preeclampsia recognizer (e.g., RBF classifier). Any combination of these parameters may provide useful information to the system. However, as described in greater detail below, certain particular features provide predictive value.

In this regard, analysis of PE versus controls resulted in a six-dimensional feature vector, including three PPG-based features and three HRV metrics per patient (Table 1). Table 1 further identifies example demographic patient data that may provide further useful information to the system and may provide further inputs to the preeclampsia recognizer facilitating improved diagnostic accuracy of example embodiments described herein.

TABLE 1

| | | | | | Pairwise Comparisons^{b} | |
|---|---|---|---|---|---|---|
| | | | | | | Preeclampsia |
| | Control | Preeclampsia | Hypertension | | Preeclampsia | vs |
| Characteristics | (n = 43) | (n = 37 ) | (n = 28) | P^{a} | vs control | hypertension |
| Maternal age, mean | 26.4 ± 5.5 | 26.6 ± 5.4 | 27.8 ± 6.1 | 0.650 | | |
| years ± SD | | | | | | |
| Body mass index, | 34.5 ± 8.9 | 33.7 ± 7.0 | 37.4 ± 9.2 | 0.254 | | |
| mean ± SD | | | | | | |
| Diastolic blood | 68.3 ± 9.0 | 86.0 ± 10.4 | 81.5 ± 8.7 | <0.001 | <0.001 | 0.11 |
| pressure, mean | | | | | | |
| mmHg ± SD | | | | | | |
| Systolic blood | 117.7 ± 1.9 | 146.2 ± 15.0 | 134.8 ± 11.4 | <0.001 | <0.001 | <0.001 |
| pressure, mean | | | | | | |
| mmHg ± SD | | | | | | |
| Gestational age, | 36.8 ± 4.3 | 32.2 ± 3.6 | 36.3 ± 3.1 | <0.001 | <0.001 | <0.001 |
| mean weeks ± SD | | | | | | |
| Race/Ethnicity, n (%) | | | | 0.732 | | |
| Caucasian | 24 (55.8%) | 18 (48.7%) | 17 (60.7%) | | | |
| African-American | 15 (34.9%) | 14 (37.8%) | 10 (35.7%) | | | |
| Hispanic | 2 (4.7%) | 3 (8.1%) | 1 (3.6%) | | | |
| Asian | 2 (4.7%) | 0 (0%) | 2 (5.4%) | | | |
| Nulliparity, n (%) | 27 (62.8%) | 18 (48.7%) | 12 (42.9%) | 0.267 | | |
| Intrauterine growth | 6 (14.0%) | 7 (18.9%) | 3 (10.7%) | 0.676 | | |
| restriction, n (%) | | | | | | |
| Cervical dilation, | 1.0 (0-4.0) | 1.0 (0-5.0) | 0 (0-3.0) | 0.139 | | |
| median cm (range) | | | | | | |
| Pain Score, median | 0 (0-8.0) | 0 (0-6.5) | 0 (0-9.0) | 0.252 | | |
| (range) | | | | | | |
| In active labor, n (%) | 20 (46.5%) | 10 (27.0%) | 9 (32.1%) | 0.173 | | |
| Antihypertensive use, | 0 (0%) | 27 (73.0%) | 10 (43.5%) | <0.001 | <0.001 | 0.065 |
| n (%) | | | | | | |
| Oxytocin | 19 (44.2%) | 8 (21.6%) | 9 (32.1%) | 0.097 | | |
| administered, n (%) | | | | | | |
| Magnesium | 3 (7.0%) | 32 (86.5%) | 5 (17.9%) | <0.001 | <0.001 | 0.064 |
| administered, n (%) | | | | | | |

^{a}P values from ANOVA or ANOVA on Ranks for continuous measures or chi-square test (calculated from logistic regression output) for categorical measures
^{b}For Pairwise comparisons, Dunnett's test was used for continuous measures and logistic regression was used for categorical measures.

Table 2 presents the mean value and standard error of the mean for the variables used in the model.

TABLE 2

| | | | P |
|---|---|---|---|
| | | | Mann- |
| | Preeclampsia | Control | Whitney |
| Features | (mean ± SD) | (mean ± SD) | U |
| Low Frequency | 799.5 ± 67.2 | 1219.2 ± 71.5 | <0.001 |
| Power (amplitude²) | | | |
| Poincare SD2 (seconds²) | 0.078 ± 0.005 | 0.099 ± 0.006 | 0.021 |
| Multiscale Entropy | 0.272 ± 0.014 | 0.349 ± 0.011 | <0.001 |
| Scales 1-5 slope | | | |
| $( \frac{dEntropy}{dScale} )$ | | | |
| Delta T (seconds) | 0.235 ± 0.007 | 0.283 ± 0.004 | <0.001 |
| Crest Time (seconds) | 0.199 ± 0.008 | 0.154 ± 0.003 | <0.001 |
| Spring Constant | 126.0 ± 10.1 | 202.8 ± 10.8 | <0.001 |
| (PPG amplitude/seconds²) | | | |

All of the variables significantly differed (P<0.001) between the two groups. The classifier used the combined contributions of all the variables to construct a classification score used for the decision.

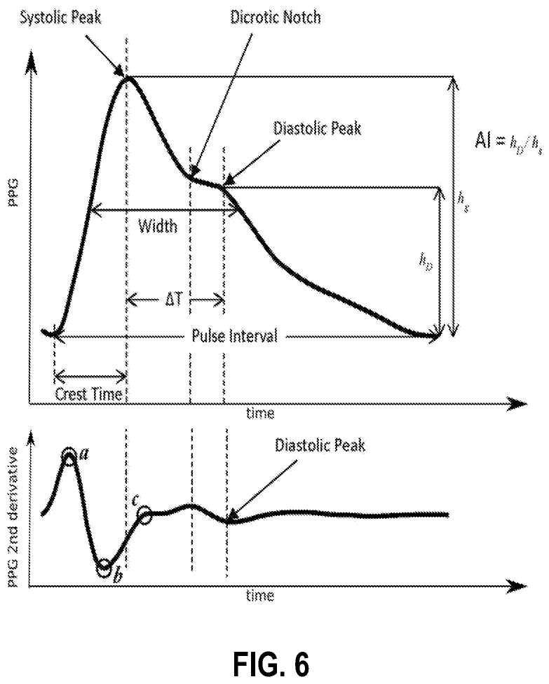

Table 3 and as also represented by FIG. 6, presents the variables used in the model discriminating PE from HTN. In this case, the pRR50, peak-to-peak interval of the PPG pulse, and variance of crest time did not individually present significant differences between the two groups, although the combination proved predictive during the feature selection procedure.

TABLE 3

| | Preeclampsia | Control | P Mann- |
|---|---|---|---|
| Features | (mean ± SD) | (mean ± SD) | Whitney U |
| pRR50 (percent) | 6.39 ± 1.85 | 12.75 ± 2.88 | 0.081 |
| Low Frequency/ | 0.193 ± 0.016 | 0.262 ± 0.024 | 0.019 |
| (Low Frequency + | | | |
| High Frequency) | | | |
| (percent) | | | |
| Peak to Peak | 0.751 ± 0.019 | 0.721 ± 0.015 | 0.395 |
| Interval (seconds) | | | |
| Variance of Crest | 4.4*10⁻⁴ ± | 9.3*10⁻⁴ ± | 0.063 |
| Time (seconds²) | 0.69*10⁻⁴ | 2*10⁻⁴ | |
| Variance of Spring | 2164 ± 351) | 5296 ± 1219 | 0.011 |
| Constant | | | |
| (PPG amplitude/ | | | |
| seconds²)² | | | |

In one embodiment, a pulse identification algorithm may be used to identify the start and end points of the individual pulses along with the systolic and diastolic peaks. To stabilize the baseline and facilitate extraction of individual features, the second derivative (as shown in FIG. 6) of the PPG signal may be taken, yielding an acceleration plethysmogram, so called because it relates to acceleration of blood in the finger. This acceleration plethysmogram may, in turn, be one of the inputs to the preeclampsia recognizer facilitating improved diagnostic accuracy of example embodiments described herein. In one embodiment, the acceleration plethysmogram provides better extraction of features from the PPG (and combination PPG/ECG signals like PTT) and also enables calculation of derivative features such as the spring constant.

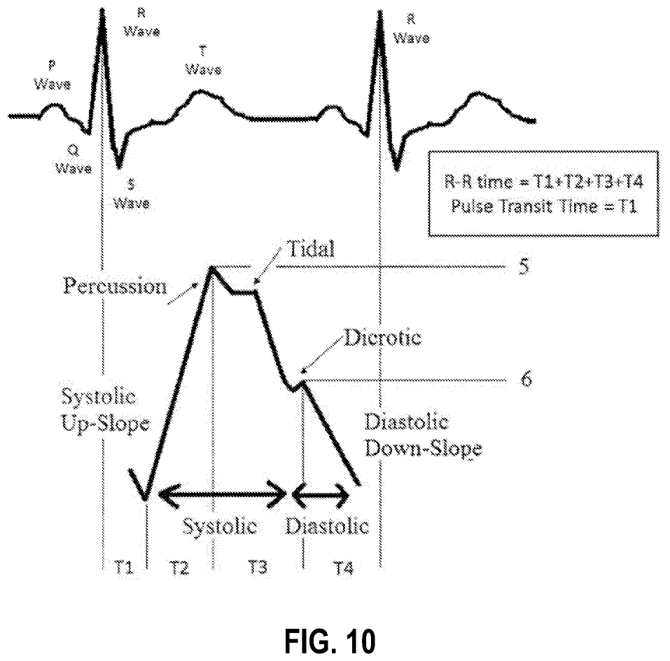

After acquiring the PPG and ECG signals, the preeclampsia recognizer (e.g., RBF classifier) finds the corresponding pulses between both signal types. From these pulses the system aggregates a multitude of relative timing features from the signals. These include timing between pulses (T1+T2+T3+T4), timing from peak of the R-wave to the dicrotic notch (T1+T2+T3), timing from the dicrotic notch to the next R-wave (T4), timing from the R-wave to first dip in the PPG signal (of pulse) (T1). Additional time and frequency features are obtained by combining subset features and applying mathematical functions (derivative, log, ratios, FFT, etc.). For example, as illustrated in FIG. 10, the heart rate is derived from 1/(average time between R waves) or (1/average 1+2+3+4)), and the pulse transit time is T1. These features are combined to create a high-dimensional feature vector that is then used in a linear or non-linear method to discern the patient types (e.g., preeclamptic patients without symptoms or clinical signs).

In one embodiment, augmentation index-like parameters are combined with pulse transit time parameters (ECG-PPG timing between ECG beat and PPG beat—how long it takes for blood to get to arm/finger) to determine whether a patient has preeclampsia, including determining whether a non-symptomatic patient (or a patient without any demonstrable clinical signs) has preeclampsia. ECG signals provide heart rate, heart rate variability, and similar parameters. Combined ECG and PPG provide PTT as described above. PTT is known to correlate with blood pressure. In certain related embodiments, PTT, in relation to heart rate variability, provides a ratio that is useful in determining a patient with preeclampsia (whether or not the patient demonstrates any symptoms or clinical signs of preeclampsia). The PPG can also be used for pulse waveshape analysis such as location of the reflective wave relative to the primary wave.

In another embodiment of the invention, the QRS peak from an ECG signal is a feature that is used to derive additional features that are applied to the high-dimensional feature vector in accordance with the subject invention. The QRS peak is used for heart rate, heart rate variability, and PTT timing. An advantage of the subject system and method is that to determine preeclampsia in a patient, neither the P or T waves of the ECG signal are required. Moreover, the finer detail of the ECG signal is also extraneous. Obtaining the QRS peak is the easiest part to capture in an ECG signal.

According to certain embodiments of the invention, combinations of timing parameters related to the feature of pulse information are features applied to a high-dimensional feature vector. For example the dicrotic notch or Pre-Ejection Period (PEP), PTT, and QRS (of the ECG) are features that can be applied to a feature vector. Other features that can apply either alone or in various combinations to a feature vector include, but are not limited to:

Time between QRS to rising slope of PPG

Time between QRS peaks

Time between dicrotic notch of the PPG and QRS peak

Time between QRS peak and the dicrotic notch of the PPG

Time between the percussion wave peak of the PPG to the QRS between pulses

Time between the rising slope of the PPG to the QRS

The height of the dicrotic notch of the PPG

The height of the percussion wave peak of the PPG

The height of the systolic wave of the PPG

Ratios of the 3 heights above

For all of these timing parameters, the mean and variance of the values are determined, as well as the “beat to beat” variability (variability of the successive differences of the parameters in the time series), before application to a feature vector.

In a pulse-oximeter, the system uses two wavelengths of light and analyzes the relationships of the two signals during the various phases of the cycle to come up with the oxygen saturation. Calculating the correct saturation requires good quality signals. Because the subject system and methods are primarily focused upon timing and secondarily on the shape of the pulse (and not saturation), a single wavelength is all that is required from a pulse-oximeter and the quality does not need to be high. Since the quality of the signal can be poor, a “reflective” sensor can be used (one that senses reflected light, versus transmitted light). Reflective sensors provide lower quality data but are more convenient since they can be used in places other than extremities (the transmitted light sensors must be used on “thin” parts of the body, like fingers, ears, noses, etc.). Accordingly, one embodiment of the invention comprises at least one optical transducer, wherein the optical transducer comprises reflective sensors.

Another embodiment of the sensor system is its ability to calculate arterial stiffness and blood pressure. These features may be used in conjunction with the preeclampsia detection system or separately.

Using the American College of Obstetricians and Gynecologists (ACOG) definition of severe preeclampsia, the system can distinguish severe preeclampsia from mild or other forms of hypertension. Severe preeclamptics require the most aggressive efforts to prevent poor outcomes or death for both the mother and fetus.

In an embodiment, particularly for high risk patients, the subject system can monitor the subject regularly (e.g. daily or weekly) or continuously and detect changes in the vascular or preterm labor status of the patient. Particularly in patients already determined likely to become preeclamptic, the system can monitor for impending symptoms or severity that would require a clinical (sometimes rapid) response. Trends in the data could be utilized to detect changes that required care such as the administration of supplements in developing nations or experimental therapies in the US. The intelligence system could be programmed with recommendations based on medical standards or previous or ongoing studies.

The system may also include methods for providing advice to the patient or clinician based on the output of the system. Methods such as fuzzy logic or rule-based systems provide the advice based on information gathered from the patient, information from clinicians, and information from the literature or standards. This information is combined by the system to provide the most relevant advice on treating the patient or preparing the patient for treatment.

The systems and methods of the invention can be used in: clinics, doctors' offices and emergency departments as a preeclampsia screening tool, in hospitals to confirm or rule-out preeclampsia in atypical presentations, and in developing nations where complications from preeclampsia are a leading cause of death, and patient transportation to an appropriate care facility poses a significant challenge. The prediction function would be invaluable in prenatal clinics for appropriate care plan development, particularly should the device predict future severe, early-onset preeclampsia in which preparation for delivery at a tertiary care center can be made. Finally, the potential for use of this device in ongoing research into prevention strategies cannot be over-stated. The ability to select only those patients destined to develop preeclampsia for clinical studies of supplements and interventions will increase the feasibility of such studies and reduce the cost of research.

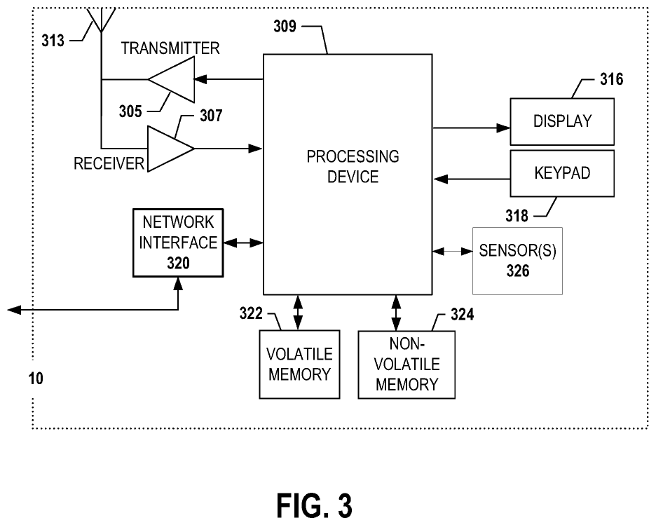

FIG. 3 provides an illustrative schematic representative of client device 10A-10N that can be used in conjunction with embodiments of the present invention. As shown in FIG. 3, a client device 10 can include an antenna 313, a transmitter 305 (e.g., radio), a receiver 307 (e.g., radio), and a processing element 309 that provides signals to and receives signals from the transmitter 305 and receiver 307, respectively, and different sensor(s) 326. Client device 10A-10N, may include or otherwise be in communication with one or more sensor(s) 326, which are worn by or otherwise associated with a patient so as to provide clinical data associated with the patient. As used herein, a patient is an individual who is being monitored for health care purposes, including monitoring performed prior to and/or following pregnancy and delivery or the like. Additionally, the patient data (also known as patient sensor data) may be any of various types of health care data collected by sensors worn by or otherwise monitoring the patient and associated with the patient so as to be utilized in conjunction with the diagnosis and monitoring of the patient for health care purposes. For example, the sensor(s) 326 may include an acceleration sensor input, skin impedance sensor, GPS location sensor, or sweat/fluid sensor. The apparatus may also include other types of sensors in other embodiments as described herein.

The signals provided to and received from the transmitter 305 and the receiver 307, respectively, may include signaling information/data in accordance with an air interface standard of applicable wireless systems to communicate with various entities, such as a preeclampsia diagnosis and classification system 30, another client device 10, and/or the like. In this regard, the client device 10 may be capable of operating with one or more air interface standards, communication protocols, modulation types, and access types. More particularly, the client device 10 may operate in accordance with any of a number of wireless communication standards and protocols. In a particular embodiment, the client device 10 may operate in accordance with multiple wireless communication standards and protocols, such as GPRS, UMTS, CDMA2000, 1×RTT, WCDMA, TD-SCDMA, LTE, E-UTRAN, EVDO, HSPA, HSDPA, Wi-Fi, WiMAX, UWB, IR protocols, Bluetooth protocols, USB protocols, and/or any other wireless protocol.

Via these communication standards and protocols, the client device 10 can communicate with various other entities using concepts such as Unstructured Supplementary Service information/data (USSD), Short Message Service (SMS), Multimedia Messaging Service (MMS), Dual-Tone Multi-Frequency Signaling (DTMF), and/or Subscriber Identity Module Dialer (SIM dialer). The client device 10 can also download changes, add-ons, and updates, for instance, to its firmware, software (e.g., including executable instructions, applications, program modules), and operating system.

According to one embodiment, the client device 10 may include location determining aspects, devices, modules, functionalities, and/or similar words used herein interchangeably. For example, the client device 10 may include outdoor positioning aspects, such as a location module adapted to acquire, for example, latitude, longitude, altitude, geocode, course, direction, heading, speed, UTC, date, and/or various other information/data. In one embodiment, the location module can acquire data, sometimes known as ephemeris data, by identifying the number of satellites in view and the relative positions of those satellites. The satellites may be a variety of different satellites, including LEO satellite systems, DOD satellite systems, the European Union Galileo positioning systems, the Chinese Compass navigation systems, Indian Regional Navigational satellite systems, and/or the like. Alternatively, the location information/data may be determined by triangulating the computing entity's position in connection with a variety of other systems, including cellular towers, Wi-Fi access points, and/or the like. Similarly, the client device 10 may include indoor positioning aspects, such as a location module adapted to acquire, for example, latitude, longitude, altitude, geocode, course, direction, heading, speed, time, date, and/or various other information/data. Some of the indoor aspects may use various position or location technologies including RFID tags, indoor beacons or transmitters, Wi-Fi access points, cellular towers, nearby computing devices (e.g., smartphones, laptops) and/or the like. For instance, such technologies may include iBeacons, Gimbal proximity beacons, BLE transmitters, NFC transmitters, and/or the like. These indoor positioning aspects can be used in a variety of settings to determine the location of someone or something to within inches or centimeters.

The client device 10 may also comprise a user interface device comprising one or more user input/output interfaces (e.g., a display 316 and/or speaker/speaker driver coupled to a processing element 309 and a touch screen, keyboard, mouse, and/or microphone coupled to a processing element 309). For example, the user output interface may be configured to provide an application, browser, user interface, dashboard, webpage, and/or similar words used herein interchangeably executing on and/or accessible via the client device 10 to cause display or audible presentation of information/data and for user interaction therewith via one or more user input interfaces. As just one specific example, the client device 10 may be configured to output various interface screens associated with a preeclampsia diagnosis and classification application, which may provide various setup/registration screens and/or may provide one or more reminder prompts for a user of the client device. The user input interface can comprise any of a number of devices allowing the client device 10 to receive data, such as a keypad 318 (hard or soft), a touch display, voice/speech or motion interfaces, scanners, readers, or other input device. In embodiments including a keypad 318, the keypad 318 can include (or cause display of) the conventional numeric (0-9) and related keys (#, *), and other keys used for operating the client device 10 and may include a full set of alphabetic keys or set of keys that may be activated to provide a full set of alphanumeric keys. In addition to providing input, the user input interface can be used, for example, to activate or deactivate certain functions, such as screen savers and/or sleep modes. Through such inputs the client device 10 can collect information/data, user interaction/input, and/or the like.

Furthermore, the network interface 320 may comprise any suitable network interface interconnecting the client device 10 with other devices operating within a larger system. For instance, the network interface 320 may include any wired or wireless communication network interface facilitating wireless communication via, for instance a local area network (LAN), personal area network (PAN), metropolitan area network (MAN), or wide area network (WAN), and/or facilitating wired communication via, for instance, a serial communication connection, standard serial buses such as, for example, RS232 or USB, SPI bus, I²C bus, or the like.

The client device 10 can also include volatile storage or memory 322 and/or non-volatile storage or memory 324, which can be embedded and/or may be removable. For example, the non-volatile memory may be ROM, PROM, EPROM, EEPROM, flash memory, MMCs, SD memory cards, Memory Sticks, CBRAM, PRAM, FeRAM, RRAM, SONOS, racetrack memory, and/or the like. The volatile memory may be RAM, DRAM, SRAM, FPM DRAM, EDO DRAM, SDRAM, DDR SDRAM, DDR2 SDRAM, DDR3 SDRAM, RDRAM, RIMM, DIMM, SIMM, VRAM, cache memory, register memory, and/or the like. The volatile and non-volatile storage or memory can store databases, database instances, database management system entities, data, applications, programs, program modules, scripts, source code, object code, byte code, compiled code, interpreted code, machine code, executable instructions, and/or the like to implement the functions of the client device 10. Again, as a specific example, the client device memory storage areas (encompassing one or both of the volatile memory 322 and/or non-volatile memory 324) may store the preeclampsia diagnosis and classification application thereon, which itself may encompass a preeclampsia model/prediction trained for predicting a likelihood of preeclampsia with severe features and providing optimal medical prompts to the user via one or more artificial intelligence and/or machine-learning algorithms. As discussed herein, a prompting strategy to be implemented with a particular user is indicative of when particular prompts are to be provided to the user. The prompting strategy may define fixed time intervals for providing prompts to the user, trigger events that may be detected prior to providing a particular prompt, and/or the like. Moreover, as discussed herein, the prompting strategy may change over time based on the results of the various machine-learning configurations as discussed herein. As one aspect that may be considered by those machine-learning configurations when determining an appropriate prompting strategy for a user, a model may be utilized to determine a predicted likelihood that the user will develop preeclampsia with severe features and adhere to a prescribed program (e.g., medication program) Similar to the prompting strategy, the model may be embodied as an artificial intelligence system (or portion of a system) taught via machine learning to provide data indicative of a predicted likelihood that the user will develop preeclampsia with severe features at some time in the future. As discussed herein, the preeclampsia diagnosis and classification application may be associated with and/or provided by an organization engaged in healthcare-related services, via the preeclampsia diagnosis and classification system 30.

In one embodiment, the client devices 10 may be configured to (independently and/or jointly with other client devices 10A-10N in the communication network 20) capture user data to be applied to the model. The client devices 10A-10N may therefore store a preeclampsia diagnosis and classification application or be in communication with one.

As shown in FIG. 4, the preeclampsia diagnosis and classification system 30 can be configured to analyze existing variables derived from the existing population which most-nearly indicates one or more characteristics associated with preeclampsia, hypertension, or preeclampsia with severe features. In certain embodiments, the preeclampsia diagnosis and classification system 30 may be embodied as a server and/or any other computing entity having one or more components as discussed above in reference to client devices 10A-10N. Moreover, the preeclampsia diagnosis and classification system 30 may be in communication (e.g., via communications network 20) with one or more client devices 10A-10N to provide a prediction of the likelihood of developing preeclampsia, hypertension, or preeclampsia with severe features as well as providing a prompting strategy for use via the preeclampsia diagnosis and classification system 30, to provide various data usable via the preeclampsia diagnosis and classification application, and/or the like. As discussed herein, the preeclampsia diagnosis and classification system 30 may be configured to continuously update and train one or more stored models, and accordingly the preeclampsia diagnosis and classification system 30 may be configured to receive data from the one or more client devices 10A-10N, and to store such data in a memory storage area in association with the preeclampsia diagnosis and classification system 30.

Currently there are no set of tests that can reliably predict the development of all cases of preeclampsia. Risk factors for preeclampsia include prior preeclampsia, chronic hypertension, multiple gestation, pregestational diabetes, high body mass index, assisted reproduction therapies, and antiphospholipid syndrome. Other factors less strongly associated with preeclampsia includes advanced maternal age, family history of preeclampsia, primiparity, etc. Gestational age, blood pressure, chest pain or dyspnea, oxygen saturation, platelet count, serum creatinine, and aspartate aminotransferase have been used in an attempt to predict severe maternal outcomes for women with early signs of preeclampsia.

Confounding diagnosis of preeclampsia is pre-existing hypertension, particularly gestational hypertension, which also presents after 20 weeks gestation. Since high blood pressure is often the first visible sign of preeclampsia, and chronic/gestational hypertension is a risk factor for preeclampsia, these pregnant patients are monitored more closely, typically requiring additional clinic visits, increasing healthcare costs. Patients with suspected preeclampsia (e.g. often times including hypertension patients) require special care to ensure their condition does not rapidly worsen and threaten the maternal or fetal well-being. Usually this involves frequent (e.g. 2 times per week) physician visits, blood pressure checks, urine tests, blood tests, and monitoring. Care can be expensive and problematic for the patient.

The preeclampsia diagnosis and classification system 30 may be further configured to analyze different data sets to differentiate between sets of diseases or severities of preeclampsia. As disclosed above, said variables or parameters were determined to best differentiate preeclampsia from hypertension. In yet another example, a different set of variables was found better suited to distinguish preeclampsia with severe features from controls.

It should be noted that there is some overlap in the parameters and, as is common in machine intelligence, different outcomes can be obtained by changing the inputs or desired outputs of the models for the preeclampsia diagnosis and classification system 30. In addition, the preeclampsia diagnosis and classification system 30 can be programmed to determine classes such as hypertension vs. preeclampsia, or it can be programmed to determine the likelihood of class (e.g. preeclampsia) as a probability or percentage. In addition, the preeclampsia diagnosis and classification system 30 can be programmed to predict the probability or likelihood of a patient in one class becoming or transitioning progressing to another class. For example, the preeclampsia diagnosis and classification system 30 may be configured to detect a potential transition to severe preeclampsia up to 12 weeks before any signs of severe preeclampsia.

In an example embodiment of the preeclampsia diagnosis and classification system 30, the preeclampsia diagnosis and classification system 30 can be used to determine whether a patient with high or rising blood pressure has or likely has preeclampsia or simply hypertension. Patients who present with high blood pressure could be triaged by the clinician or staff using this non-invasive, low cost device before elevating the care level that results in increased costs and decreased quality of life for the mother. Preferentially, the preeclampsia diagnosis and classification system 30 would be programmed to create a “rule-out preeclampsia” test that has a very low number of false negatives. The preeclampsia diagnosis and classification system 30 is configured to implement a rule-out method by training an algorithm (or set the threshold for the algorithm) such that it never or almost never creates false negatives. Additionally or alternatively, the preeclampsia diagnosis and classification system 30 is configured to train the algorithm to minimize the false positives, which would provide the opposite type of predictor (rule-in) that would determine that this group of patients having these variables has preeclampsia. For example, a rule-out preeclampsia test may function in such a way that 100 patients are tested and the test results in 2 groups or classifications with 50 patients in each group. The first group is the negative group, and of this group of 50 patients, none will eventually have preeclampsia. The second group is the positive group, and 20 of the 50 patients end up with preeclampsia. As such, it isn't very good at predicting whether someone gets preeclampsia or not (40%) but it is very good at predicting that a patient won't get preeclampsia (50/50). So using rule-out preeclampsia test allows medical professionals to quickly determine that 50% of the patients do not need workup for preeclampsia.

At least one benefit of the rule-out preeclampsia test is that it is an inexpensive and easy to use method that can rule-out preeclampsia on even half the patients. This leads to preeclampsia work-up costs cut in half. In addition, the preeclampsia diagnosis and classification system 30 may be used continuously by the patient (either worn daily or used each morning or evening) with data transmitted to a clinician automatically or stored for download by the clinician in an upcoming appointment. The preeclampsia diagnosis and classification system 30 may be programmed to detect the earliest onset of preeclampsia and provide an alert or indicate that the patient should see a clinician as soon as possible. Additionally or alternatively, the preeclampsia diagnosis and classification system 30 may be configured to request that the patient use a blood pressure cuff or other home medical device to confirm the trend towards preeclampsia (for example, via increasing blood pressure or increased protein in the urine), before contacting the clinician. In yet another embodiment, the preeclampsia diagnosis and classification system 30 may be programmed to determine the likelihood that the patient with hypertension would become preeclamptic during the pregnancy. As such, the preeclampsia diagnosis and classification system 30 would be useful in limiting the number of tests required of patients who are very unlikely to become preeclamptic.

In another embodiment, the preeclampsia diagnosis and classification system 30 may be programmed, usually with different features or parameters than in the previous embodiments, to determine the difference between mild preeclampsia and severe preeclampsia. Mild preeclampsia often can be managed as an outpatient until the baby reaches maturity, whereas severe features often necessitate immediate delivery, even when the baby is preterm. As such, determining the presence of severe features rapidly and inexpensively, can greatly improve patient care. In addition, as discussed above, predicting the probability that a patient will move from mild to severe can be very important and lead to better, more efficient patient care—with the patients at highest risk undergoing frequent monitoring and care. In one example, the preeclampsia diagnosis and classification system 30 could be used to obtain early warning of problems for patients at high risk, allowing an increased number of patients to go home safely and not remain confined to the hospital.

In another embodiment, in addition to using the ECG and PPG data, the preeclampsia diagnosis and classification system 30 also uses maternal/fetal demographics and/or risk factors to facilitate risk assessment and clinical decision support. Based on the values of the ECG and PPG models and the maternal/fetal demographics and/or risk factors, blood pressure, proteinuria, and/or other physiologic data, a more accurate model is created that can predict the likelihood of hypertension, preeclampsia, severe preeclampsia, or poor or good maternal/fetal outcomes. In addition, recommendations can be provided by the preeclampsia diagnosis and classification system 30 based on the overall risk profile of the patient including treatment plans, supplements, and other clinical decision support. For example, the preeclampsia diagnosis and classification system 30 may be configured to recommend the use of aspirin, calcium, magnesium and/or a visitation schedule based on the overall risk profile of the patient.

In this way, the preeclampsia diagnosis and classification system 30 may support multiple algorithms and methodologies, including those discussed herein with respect to monitoring, determining, and managing preeclampsia, analyzing the data together with existing and/or learned data to develop a care management and adherence strategy to ensure the health of the patient and baby.

In some embodiments, with reference to FIG. 4, the preeclampsia diagnosis and classification system 30 may include a preeclampsia recognizer 208, a data training engine 404, and a model selector 406, all of which may be in communication with a database (not shown). The preeclampsia diagnosis and classification system 30 may receive one or more patient data and may generate the appropriate assessment that can reliably predict the development of all cases of preeclampsia using statistics and machine learning. The preeclampsia diagnosis and classification system 30 may use any of the algorithms, operations, steps, and processes disclosed herein for receiving or capturing patient data and context information, generating the appropriate assessment that can reliably predict the development of all cases of preeclampsia, and generating notifications to encourage medication adherence and apply patient care.

The preeclampsia diagnosis and classification system 30 may receive a plurality of inputs 402 from one or more client devices 10A-10N and process the inputs 402 within the preeclampsia diagnosis and classification system 30 to produce an output via an output generator 408, which may include appropriate preeclampsia assessment and medical care strategies to improve maternal outcomes for patients with early signs of preeclampsia. In some embodiments, the preeclampsia diagnosis and classification system 30 may extract patient data and environment data using the preeclampsia recognizer 208, and input the extracted data into a model to produce an output from the model regarding the patient data. The output comprises a prediction of whether the patient will develop any of the classes of preeclampsia (e.g., mild vs severe) or hypertension. The preeclampsia diagnosis and classification system 30 may further train said models and/or medical adherence strategies. The preeclampsia diagnosis and classification system 30 may further comprise a model selector 406 which is configured to select a model from a plurality of trained models trained to identify and diagnosis whether the patient has hypertension, preeclampsia, or preeclampsia with severe features. Thereafter, the preeclampsia recognizer may use the selected model to generate the predicted development of preeclampsia, transmit updates to the models, and output the results via the output generator 408 which may, in turn, be configured to output one or more automated-prompting notifications via any suitable client devices 10A-10N. Similarly, the model selector 406 may select from rule-out or rule-in versions of the models based on the goals of the user (patient or clinician).

When inputs 402 are received by the preeclampsia diagnosis and classification system 30, patient data is extracted from two or more electrodes and one or more optical transducers as part of the client devices 10A-10N using the preeclampsia recognizer 208. The patient data includes such information as current and past patient medical history, likelihood of developing preeclampsia, and what type of medical treatment is needed to prevent the development of preeclampsia with severe features.

The preeclampsia diagnosis and classification system 30 may then compute the output using the preeclampsia recognizer 208, data training engine 404, and the model selector 406. The data training engine 404 draws information about the patient data, environmental data, sensor data, prediction data from an initial set of training data to train the models. In one embodiment, the initial set of training data comprises training data where certain signals or features are determined to be best differentiate target classes of diagnosis or detection of hypertension, preeclampsia, or preeclampsia with severe features for the patient. For example, in considering features distinguishing preeclampsia from normotensive controls, the following features related to PPG and HRV are considered:

PPG features: (1) Delta T—The time between the systolic and diastolic peaks. Reflects the time required for the pulse to propagate from the heart to the periphery and back; (2) Crest Time—The time from the beginning of the pulse to the systolic peak. Has been used as a surrogate for the pulse velocity; and/or (3) Spring Constant—A surrogate measure for the pulse wave velocity. Its derivation is based on modeling of the pressure wave and its interaction with the arterial walls.

HRV features: (1) Low Frequency—Low frequency power reflects sympathetic activity with some influence from the parasympathetic nervous system; (2) PoincareSD2N—The standard deviation in the second principal direction of the plot; Poincare plots are a widely used technique that describes inter-beat interval fluctuations; and (3) Slope of Sample Entropy values for Scales 1 through 5—The slope of the Multiscale Sample Entropy for the scales 1 through 5. Sample Entropy quantifies the irregularity of a time series and estimates the probability that two time series that are similar (within a tolerance) will remain similar when another point is added in the time series. Used to calculate the slope of the curve for the scales 1 through 5 and the slope for the scales 6 through 20, which represent the heartbeat dynamics for the high and low frequencies, respectively.

In considering features distinguishing preeclampsia from hypertension controls, the following features related to PPG and HRV are considered in the initial training set: PPG features: (1) Peak to Peak Interval—The time between successive photoplethysmographic systolic peaks. Associated with arterial stiffness; (2) Variance of the Crest Time; and (3) Variance of the Spring Constant; HRV features: (1) pRR50—The percent of pairs of R-R intervals whose difference exceed 50 ms reflects the short-term variations in the duration of the R-R interval; and (2) Normalized Low Frequency (LF)—The percent of the LF component over the total power of the heart rate variability.

The data training engine 404 is then further configured to implement the following classification/model process steps of partitioning the initial set of training data into a plurality of training data sets and a plurality of test data sets derived from the patient data using multi-fold cross-validation, wherein extracting the features of the training data and test data comprises extracting the features of each fold of the training data and each fold of the test data, and wherein training the model comprises training the model for each fold, and wherein testing the model comprises testing the model for each fold.

In light of the data training engine's 404 classification simulations, the model selector 406 selects the model trained to identify the following conditions: normotensive pregnancies, patients with hypertension, preeclampsia with mild features, and/or preeclampsia with severe features. The selected model may then be transmitted by the preeclampsia diagnosis and classification system 30 to the sensor device 200 for updating the local instance of the preeclampsia recognizer so as to better predict the user's most likely condition over time and associate the user's current medical status and context to strategize a medical treatment plan.

In some embodiments, care and management of preeclampsia includes monitoring and managing blood pressure and frequent tests to ensure the health of the mother and baby. Patients with preeclampsia that is believed to be stable and that are expected to be reliable in reporting problems and measuring blood pressure may be treated as outpatients. The preeclampsia diagnosis and classification system 30 enables patients with high risk of preeclampsia or hypertensive diseases of pregnancy to be more reliably monitored at home. The preeclampsia diagnosis and classification system 30 would include a kit with a smart phone application that uses data from the preeclampsia ECG/PPG sensor device, a blood pressure monitor, proteinuria tests, patient information and feedback (e.g. presence of headaches, weakness, abdominal pain, etc.), and/or home blood test methodologies to collect data and reliably transfer this data back to the clinician. Additionally, the application would remind the woman to implement the tests at the required intervals, enter the data, including regular questionnaires, and/or home blood pressure checks.

The preeclampsia diagnosis and classification system 30 may be configured to estimate the patient's likelihood to develop all cases of preeclampsia. In some example embodiments, the data training engine 404 using trained predictive models selected by the model selector 406 may be configured to estimate forecast preeclampsia with severe features via one or more statistics and/or machine learning algorithms. The artificial intelligence engine may provide a plurality of algorithms, which may utilize a least absolute shrinkage and selection operator (LASSO) method. LASSO is a popular and attractive technique for variable selection for high-dimensional data. It uses a regularization technique to select the parameters most likely to create a good model of the data and has been shown to work very well for problems with many variables and limited sample sizes.

With the model selector 406 configured to provide models for quantifying a user's predicted likelihood to develop preeclampsia with severe features over time, various artificial intelligence learning techniques may be utilized to train the model to optimize future predictions and medical treatment strategies.

The preeclampsia diagnosis and classification system 30 may also determine that the parameters from the ECG and PPG can be used to monitor changes in blood pressure. Parameters such as pulse transit time are correlated with blood pressure, but often times it is difficult to determine the actual blood pressure. By monitoring and trending pulse transit time or similar features of the ECG and PPG, changes in blood pressure can be detected. Using this technique, it may be possible to reduce the number of times the patient takes their blood pressure, or to more accurately detect unexpected times of high blood pressure.

In addition to careful monitoring, patients with suspected preeclampsia or hypertensive diseases of pregnancy should exercise regularly during pregnancy to maintain their health and body weight, reducing the likelihood of hypertension. As such, another embodiment of the preeclampsia diagnosis and classification system 30 would include features of a fitness tracking device (e.g., Fitbit) to measure activity levels and heart rate of the patient. Some of these features could be easily determined by the ECG and PPG features of the preeclampsia diagnosis and classification system 30, others require the addition of an accelerometer, GPS sensor, impedance monitoring, or similar sensors to determine and monitor appropriate exercise levels for the patient. In addition, the preeclampsia diagnosis and classification system 30 would request daily updates on the weight of the patient, either directly from the patient or via a connected scale, for tracking weight gain.

As will be appreciated, one or more of the preeclampsia diagnosis and classification system 30 components may be located remotely from other components, such as in a distributed system. Furthermore, one or more of the components may be combined and additional components performing functions described herein may be included in the preeclampsia diagnosis and classification system 30. Thus, the preeclampsia diagnosis and classification system 30 can be adapted to accommodate a variety of needs and circumstances.

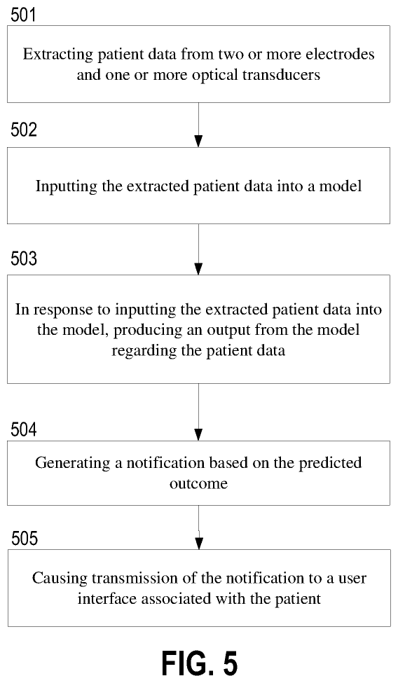

FIG. 5 specifically illustrates an example flowchart providing various operations, steps and processes for predicting a likelihood of preeclampsia with severe features for a particular user, via the preeclampsia diagnosis and classification system 30 and/or the one or more client devices 10A-10N or other user-specific devices. Via the various steps of FIG. 5, a user's preeclampsia classification may be determined in real-time or near real-time with the preeclampsia diagnosis and classification system 30 and/or the client devices 10A-10N interacting with the user (e.g., via one or more devices providing prompts to the user and/or generating electrodes and transducer(s) sensor data regarding the user).

As indicated at Block 501 of FIG. 5, the preeclampsia diagnosis and classification system 30 and/or various client devices 10A-10N begin extracting patient data from two or more electrodes and one or more optical transducers. In certain embodiments, the patient data includes activity sensors (e.g. accelerometers, actigraphy, etc.) which provide information about the patient's exercise and activity levels, helping the mother maintain fitness. Reminders, daily activity goals, and other common fitness monitoring techniques can be used to help the mother maintain a healthy active lifestyle. Patient data may further include measuring temperature to prevent overexertion and detect fever. Fetuses cannot expel heat well and therefore it is important that the mother keep her body temperature in a safe range (e.g. 97-100 degrees F.). Additionally, measuring hydration levels with skin sensors, impedance or patient feedback (e.g. how much water did you drink today) protects the baby from dehydration. Dehydration can harm the mother and the baby and in some cases can cause preterm labor. Activity levels, ambient temperature data, and fluid intake can also be used to track adequate hydration and help the mother maintain appropriate levels.

Patient data related to overexertion can also be captured and monitored by heart rate. Exercise is important during pregnancy to maintain healthy weight and body, but overexertion can harm the mother and the fetus. Maintaining a safe heart rate can help prevent overexertion. The PPG/ECG or similar heart rate sensors can be utilized to monitor heart rate continuously and warn the patient if she is over doing it. Additionally, plots can be generated by the preeclampsia diagnosis and classification system 30 and/or the client devices 10A-10N each day indicating where the mother may have had too high a heart rate.

As described above, regular checking of blood pressure using the preeclampsia diagnosis and classification system 30 and/or the client devices 10A-10N can help determine when hypertension or hypertensive diseases of pregnancy may be starting. Similarly, long term changes in heart rate or heart rate variability may be indicative of physiologic changes that may be tracked or reported by the preeclampsia diagnosis and classification system 30 and/or the client devices 10A-10N.

Other patient related data including sleep apnea, periodic cessation of breathing during sleep, can cause problems for both the mother and fetus and is often associated with or worsened by pregnancy. Sleep apnea can be monitored with many sensors including microphones (listening for snoring), actigraphy (detecting arousals), PPG (detecting changes in vascular volume associated with increased breathing efforts), and ECG variations provided by the preeclampsia diagnosis and classification system 30 and/or the client devices 10A-10N. Reporting possible sleep apnea to the patient or clinician can help avoid potential harm.

As discussed in greater detail in reference to Block 502, below, the preeclampsia diagnosis and classification system 30 is further configured to input the extracted patient data into a model.

As indicated at Block 503 of FIG. 5, in response to inputting the extracted patient data into the model, produce an output from the model regarding the patient data.

As a part of the training process discussed herein, the preeclampsia diagnosis and classification system 30 may be configured to train the model based on an initial set of training data, wherein the data training engine is further configured to train the model by: partitioning the initial set of training data into training data and test data; extracting features of the training data and test data; training the model using the training data; and testing the model using the test data. For example, the preeclampsia diagnosis and classification system 30 is configured to extract features of the training data and the test data by applying a least absolute shrinkage and selection operator (LASSO) procedure to the training data and the test data to identify the features for extraction and extracting the features in response to applying the LASSO procedure.

The preeclampsia diagnosis and classification system 30 may be configured to discriminate patients with normotensive pregnancies, hypertension, preeclampsia with mild features, and preeclampsia with severe features by selecting the model that best identifies said conditions. For example, the preeclampsia diagnosis and classification system 30 may determine the performance measurement of each model and select the model based on a performance measurement of each model in comparison with the other models.

Moreover, the preeclampsia diagnosis and classification system 30 and/or the one or more client devices 10A-10N may be configured to generate a notification based on the predicted outcome and cause transmission of the notification to a user interface associated with the patient as shown in Blocks 505 and 506.

In certain embodiments, causing transmission of the notification to the user interface associated with the patient comprises at least one of: (i) causing transmission of the notification to a user interface of the apparatus; (ii) causing transmission of the notification to a patient's user device; or (iii) causing transmission of the notification to a doctor's user device.

In certain embodiments, the preeclampsia diagnosis and classification system 30 may be configured to perform one or more actions related to fetal and maternal health. For example, the preeclampsia diagnosis and classification system 30 may provide fetal and maternal health related advice and data recording to be provided to a healthcare provider (e.g., monitor and track baby kicks as an important indicator of fetal health) such that the healthcare provider may apply suitable action or advice. In one embodiment, a simple technique of tapping the wrist band of the client devices 10A-10N every time a kick occurs provides an easy method of tracking the mother's sensation of kicks. The wrist band or watch mechanism, for example device 200, would include a button or touch screen that could be tapped easily, perhaps also including a message to verify that the mother detected a kick. Additionally, with voice recognition software, the mother could verbally indicate that they felt a kick (e.g., “Alexa, I felt a kick”).

In addition to sensors and mechanisms for tracking and monitoring maternal and fetal well-being, the preeclampsia diagnosis and classification system 30 could provide advice and recommendations based on a variety of information including gestational age, risk factors, maternal characteristics, physiologic inputs, etc. The device could recommend daily Kegel exercises. The preeclampsia diagnosis and classification system 30 could track and monitor and recommend an increase or decrease in daily activities.

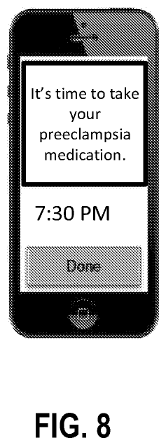

The preeclampsia diagnosis and classification system 30 would also remind the mother to take her prenatal vitamins, preeclampsia medications, blood pressure medications, and other medications as shown in FIG. 8. The preeclampsia diagnosis and classification system 30 could further transmit a query to the mother on whether she took her medications. In another embodiment, the preeclampsia diagnosis and classification system 30 may be configured to track her medication adherence with electronic pills or capsules, smart packaging (bottles or punch outs), or other monitoring methods.

The preeclampsia diagnosis and classification system 30 could also provide advice, warnings, reminders, and feedback to help the mother deal with her pregnancy. For example, the preeclampsia diagnosis and classification system 30 may transmit a reminder to the mother with “you are now entering your third trimester, your baby is now 12 inches long and normally weighs approximately 2 pounds.”

Moreover, the patient or mother's adherence may be tracked via the preeclampsia diagnosis and classification system 30 that enables a health provider to have visibility into the mother's adherence with a prescribed medicinal therapy and/or exercise regimen. Thus, upon detecting a particularly low rate of adherence via the preeclampsia diagnosis and classification system 30, the health provider may appropriately adjust the prescribed medicinal therapy and/or exercise regimen for the user to compensate for the prior low adherence rates.

The following study was conducted to validate the ability of the system and method of the invention to identify preeclampsia in a patient. After written, informed consent, 66 women admitted to Labor & Delivery were studied with the distribution shown in the table below.

| Diagnosis | Average GA | N |
|---|---|---|
| Control | 36.2 | 27 |
| Gestational Hypertension | 38.3 | 4 |
| Chronic Hypertension | 33.9 | 9 |
| Chronic Hypertension with | 31.4 | 7 |
| Super-Imposed | | |
| PreEclampsia | | |
| PreEclampsia | 33.1 | 19 |

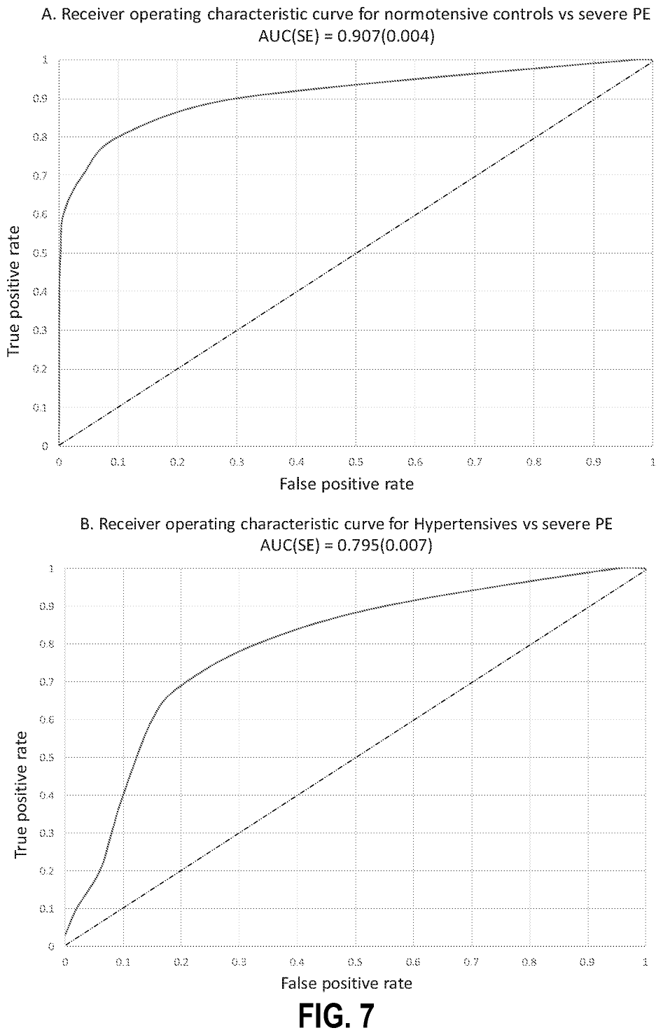

Continuous ECG recording from the maternal chest and pulse oximetry waveforms (photoplethysmography, PPG) from the middle finger were obtained for 30-minutes with the patient at rest. Various timing features were obtained from each data set relative to the PPG and ECG signals. These features were then used as input into a Radial Basis Function Network (RBF) with a linear output to discriminate/detect preeclamptics versus controls. The RBF was trained with 1000 different trials utilizing different mixtures of training and cross validation data. The sensitivity of the system was 0.86, the PPV was 0.75, and the area under the curve (AUC) of the receiver operating characteristic (ROC) curve was 0.8 as shown in FIG. 7. The combination of sensitivity and PPV is superior to any other research reported to date (excluding invasive, chemical, or biomarker methods) and has been achieved using a simple, inexpensive pulse-oximeter and ECG lead.

Simultaneously, antenatal data in the high risk OB clinic was collected. Inclusion criteria consist of women prior to 25 weeks gestation with multi-fetal gestation, chronic hypertension, pre-gestational diabetes, or history of preeclampsia in a prior pregnancy. After written informed consent, subjects underwent the same protocol as above for 30-minutes at each prenatal visit and again when they presented for delivery, if possible. Data was stored for subsequent analysis in light of delivery outcome. To date 26 women have enrolled. Of those, 11 have delivered: 7 with preeclampsia and 4 without. Using the term patients (control and preeclamptics described above) to train the RBF predictive model, 82% of subjects were correctly predicted at least 10 weeks before the onset of symptoms (or delivery). The RBF was trained with 1000 different trials utilizing different mixtures of training and cross validation data. The sensitivity of the system was 0.86, the PPV was 0.75, and the area under the curve (AUC) of the receiver operating characteristic (ROC) curve was 0.8. The combination of sensitivity and PPV is superior to any other research reported to date (excluding invasive, chemical, or biomarker methods) and has been achieved using a simple, inexpensive sensor device comprising a pulse-oximeter and ECG lead.

Many modifications and other embodiments will come to mind to one skilled in the art to which this disclosure pertains having the benefit of the teachings presented in the foregoing descriptions and the associated drawings. Therefore, it is to be understood that the disclosure is not to be limited to the specific embodiments disclosed and that modifications and other embodiments are intended to be included within the scope of the appended claims. Although specific terms are employed herein, they are used in a generic and descriptive sense only and not for purposes of limitation.

We claim:

1. An apparatus for diagnosis and classification of preeclampsia-related conditions, the apparatus comprising:
a preeclampsia recognizer configured to:
extract patient data from two or more electrodes and one or more optical transducers attached to a patient;
input the extracted patient data into a model;
in response to inputting the extracted patient data into the model, produce an output from the model regarding the patient data;
generate a notification based on the predicted outcome; and
cause transmission of the notification to a user interface associated with the patient.

2. The apparatus of claim 1, further comprising:
a data training engine programmed to train the model based on an initial set of training data.

3. The apparatus of claim 2, wherein the data training engine is further configured to train the model by:
partitioning the initial set of training data into training data and test data;
extract features of the training data and test data;
train the model using the training data; and
test the model using the test data.

4. The apparatus of claim 3, wherein partitioning the initial set of training data comprises:
partitioning the initial set of training data into a plurality of training data sets and a plurality of test data sets using multi-fold cross-validation,
wherein extracting the features of the training data and test data comprises extracting the features of each fold of the training data and each fold of the test data,
wherein training the model comprises training the model for each fold, and
wherein testing the model comprises testing the model for each fold.

5. The apparatus of claim 4, wherein extracting features of the training data and the test data comprises:
applying a least absolute shrinkage and selection operator (LASSO) procedure to the training data and the test data to identify the features for extraction; and
extracting the features in response to applying the LASSO procedure.

6. The apparatus of claim 1, further comprising:
a model selector configured to select the model from a plurality of models, wherein the plurality of models are trained to identify corresponding specific conditions.

7. The apparatus of claim 6, wherein the specific conditions comprise normotensive pregnancies, patients with hypertension, preeclampsia with mild features, and preeclampsia with severe features.

8. The apparatus of claim 6, wherein the model selector is configured to select the model in response to input from a user or based on a performance measurement of each model of the plurality of models.

9. The apparatus of claim 8, wherein a data training engine is configured to:
determine the performance measurement of each model of the plurality of models,
wherein selection of a model based on the performance measurement of each model comprises selecting a best performing model.

10. The apparatus of claim 1, wherein the two or more electrodes and the one or more optical transducers are co-located in a single sensor device.

11. The apparatus of claim 1, wherein the two or more electrodes and the one or more optical transducers are located in separate sensor devices.

12. The apparatus of claim 1, wherein the one or more optical transducers are located in a pulse oximeter.

13. The apparatus of claim 1, wherein the patient data comprises a set of possible variables including one or more of: heart rate, pulse transit time, augmentation indices, variability of heart rate, variability of pulse transit time, variability of augmentation indices, and combinations or ratios of the aforementioned possible variables.

14. The apparatus of claim 13, wherein the patient data further comprises a movement of the patient, an activity of the patient, an action of the patient, a schedule of the patient, a weight of the patient, a temperature of the patient, or a hydration level of the patient.

15. The apparatus of claim 1, wherein the model differentiates between mild and severe preeclampsia.

16. The apparatus of claim 1, further comprising:
a sensor device comprising the two or more electrodes and the one or more optical transducers, wherein the sensor device is portable and/or wearable.

17. The apparatus of claim 1, wherein causing transmission of the notification to the user interface associated with the patient comprises at least one of:
(i) causing transmission of the notification to a user interface of the apparatus;
(ii) causing transmission of the notification to a patient's user device; or
(iii) causing transmission of the notification to a doctor's user device.

18. The apparatus of claim 1, wherein the preeclampsia recognizer is further configured to:
extract patient data indicative of physical activity data of the patient;
input the extracted patient data indicative of physical activity data of the patient into the model; and
in response to inputting the extracted patient data into the model, determine a diagnosis of preeclampsia, preeclampsia with severe features, or hypertension.

19. A computer-implemented method for diagnosing and classifying preeclampsia-related conditions in a patient comprising steps of:
extracting patient data from two or more electrodes and one or more optical transducers attached to a patient;
inputting the extracted patient data into a model;
in response to inputting the extracted patient data into the model, producing an output from the model regarding the patient data;
generating a notification based on the predicted outcome; and
causing transmission of the notification to a user interface associated with the patient.

20. A portable device for diagnosis and classification of preeclampsia-related conditions, the portable device comprising:
two or more electrodes,
one or more optical transducers,
memory to store computer readable instructions and data; and
a processor configured to access the memory and execute the computer readable instructions to:
extract patient data from two or more electrodes and one or more optical transducers attached to a patient;
input the extracted patient data into a model;
in response to inputting the extracted patient data into the model, produce an output from the model regarding the patient data;
generate a notification based on the predicted outcome; and
cause transmission of the notification to a user interface associated with the patient.
